(19) United States
(12) Patent Application Publication
(10) Pub. No.: US 2020/0069644 A1
(43) Pub. Date: Mar. 5, 2020

(54) NOVEL USE OF A FORMYL PEPTIDE RECEPTOR 2/ LIPOXIN A4 RECEPTOR (FPR2/ALX) AGONIST FOR TREATMENT OF HEART FAILURE

(71) Applicant: BRISTOL- MYERS SQUIBB COMPANY, Princeton, NJ (US)

(72) Inventors: Jacek Ostrowski, Jamison, NJ (US); Ricardo Garcia, Ewing, NJ (US); Nicholas R. Wurtz, Pennington, NJ (US); Nancy Lersey CARSON, Nationl Park, NJ (US)

(21) Appl. No.: 16/612,438

(22) PCT Filed: May 22, 2018
(86) PCT No.: PCT/US2018/033783

§ 371 Date: Nov. 11, 2019

Related U.S. Application Data
(60) Provisional application No. 62/509,489, filed on May 22, 2017.

(51) Int. Cl. A61K 31/4015; A61K 45/06; A61P 9/04

(52) U.S. Cl. CPC A61K 31/4015; A61P 9/04; A61K 45/06

(57) ABSTRACT

The disclosure generally relates to methods of treating heart failure with Compound 1, 1-((3S,4R)-4-(2,6-difluoro-4-meth oxyphenyl)-2-oxopyrrolidin-3-yl)-3-phenylurea.

### CROSS REFERENCE TO RELATED APPLICATIONS

This application is entitled to priority pursuant to 35 U.S.C. § 119(e) to U.S. provisional patent application No. 62/509,489, filed May 22, 2017, which is incorporated herein in its entirety.

### BACKGROUND OF THE INVENTION

Heart disease is an increasingly prevalent condition that exerts a significant clinical and economic burden. The increase in prevalence is driven in part by patients surviving myocardial infarctions leading to cumulative myocardial damage that progressively leads to adverse cardiac remodeling and left ventricular dysfunction (Viau D M et al., Heart, 2015, 101, 1862-7, Paulus W J., Tschope C., J. Am. Coll. Cardiol., 2013, 62, 263-71). Heart disease by etiologies that are non-ischemic in nature such as dilated cardiomyopathy, myocarditis and chronic hypertension add to the increased prevalence of heart failure. In the U.S., heart failure (HF) affects over 5 million people with approximately half a million new cases occurring each year. HF is the leading cause of hospitalizations in people over 65 years in age. HF has many potential causes and diverse clinical features. Symptoms of heart failure can include dyspnea during activity or at rest, cough with white sputum, rapid weight gain, swelling in ankles, legs and abdomen, dizziness, fatigue and weakness, rapid or irregular heartbeats, nausea, palpitations, and chest pains.

Ischemic and non-ischemic cardiomyopathies are two distinct types of heart disease that can lead to HF. Ischemic HF describes significantly impaired left ventricular function that results from reduced blood supply to the heart muscle, commonly from coronary artery disease. In contrast, non-ischemic HF has a range of etiologies, including congenital, infectious agents, autoimmune, and idiopathic causes. Identifying molecular differences between ischemic and non-ischemic HF may reveal new etiology-specific treatments.

Clinically, HF may be divided into two primary subsets, diastolic heart failure (DHF) and systolic heart failure (SHF). SHF, which is also known as heart failure with reduced ejection fraction (HF_{R}EF), involves an abnormality of the heart resulting in failure of the heart to pump blood at a rate needed for metabolizing tissues at rest and/or during exertion. DHF, which is also known as heart failure with preserved ejection fraction. (HF_{P}EF), is a clinical syndrome with symptoms and signs of HF. Patients with HF_{P}EF show declined performance of a heart ventricle, not at the time of contraction (systole), but during the phase of filling (diastole). HF_{P}EF patients show normal ejection fraction of blood pumped out of the ventricle, but the heart muscle does not quickly relax to allow efficient filling of blood returning from the body. The clinical manifestations of HF_{R}EF and HF_{P}EF have distinct differences in risk factors, patient characteristics, and pathophysiology. Moreover, medications proven effective in HF_{R}EF have not been found to be effective in HF_{P}EF. At present there are no approved treatments to reduce mortality in HF_{P}EF even though about half of heart failure patients have heart failure with preserved ejection fraction (HF_{P}EF). Consequently, there remains a need for finding pharmaceutical agents useful for treating and preventing HF_{P}EF.

Formyl peptide receptor 2/lipoxin A4 (FPR2/ALX) belongs to small group of seven-transmembrane domain, G protein-coupled receptors that are expressed mainly by mammalian phagocytic leukocytes and are known to be important in host defense and inflammation. In the cardiovascular system, both the FPR2/ALX receptor and its pro-resolution agonists were found to be responsible for atherogenic-plaque stabilization and healing (Petri M H., et al., Cardiovasc. Res., 2015, 105, 65-74; and Fredman G., et al., Sci. Trans. Med., 2015, 7(275); 275ra20). Lipoxin and FPR2 have also shown benefit in preclinical models of chronic inflammatory human diseases, including: infectious diseases, psoriasis, dermatitis, ocular inflammation, sepsis, pain, metabolic/diabetes diseases, cancer, COPD, asthma and allergic diseases, cystic fibrosis, acute lung injury and fibrosis, rheumatoid arthritis and other joint diseases, Alzheimer's disease, kidney fibrosis, and organ transplantation (Romano M., et al., Eur. J. Pharmacol., 2015, 5, 49-63, Perrett, M., et al., Trends in Pharm. Sci., 2015, 36, 737-755.)

Recently, there are a number of published reports that describe the use of endogenous pro-resolution eicosanoids and peptides in the setting of myocardial infarction (MI) (Dalli et al., Proresolving and tissue-protective actions of annexin A1-based cleavage-resistant peptides are mediated by formyl peptide receptor 2/lipoxin A4 receptor. J. Immunol. 2013 Jun. 15; 190(12):6478-87; Gobbetti et al., Nonredundant protective properties of FPR2/ALX in polymicrobial murine sepsis. Proc Natl Acad Sci USA. 2014 Dec. 30; 111(52):18685-90; Heo et al., Formyl peptide receptor 2 is involved in cardiac repair after myocardial infarction through mobilization of circulating angiogenic cells. Stem Cells. 2017 March; 35(3):654-665; Kain et al., Resolvin D1 activates the inflammation resolving response at splenic and ventricular site following myocardial infarction leading to improved ventricular function. J Mol Cell Cardiol. 2015 July; 84:24-35; Kain et al, Resolution agonist 15-Epi-Lipoxin A4 directs FPR2 to expedite healing phase post-myocardial infarction FASEB J, 2016 30 (1) S306.2; Perretti et al., Characterizing the anti-inflammatory and tissue protective actions of a novel Annexin A1 peptide. PLoS One. 2017 Apr. 13; 12(4)). These studies demonstrate that the resolution pathway can be induced at the time of a surgically induced MI to improve cardiac structure and function in the mouse. These studies focus on the effects of acute prevention therapy where test article is given either before myocardial infarction or at the time on myocardial infarction. Acute treatments were given as a single administration or over the course of several days post myocardial infarction. Moreover, these studies all use the parenteral route for administration of test article (i.e., intraperitoneal or intravenous). To date, there is only one report of a synthetic small-molecule pro-resolution agonist used in the setting of myocardial infarction (Qin et al., Small-molecule-biased formyl peptide receptor agonist compound 17b protects against myocardial ischaemia-reperfusion injury in mice. Nat Commun. 2017 Feb. 7; 8:14232). The compound was given once via IP injection 1 day before myocardial infarction and showed improvement in infarct structure and cardiac function. In these reports, the benefits of stimulating pro-resolution pathways are all exemplified in models that drive towards ischemic heart disease.

Unlike the foregoing studies, in which FPR2 agonists were either dosed before the MI was induced or dosed acutely immediately post MI, the compound of the present invention, 1-((3S,4R)-4-(2,6-difluoro-4-methoxyphenyl)-2-oxopyrrolidin-3-yl)-3-phenylurea (a FPR2 agonist hereinafter referred to as Compound 1), was given as a chronic therapy via oral administration for several weeks and showed improvement in multiple heart failure models of ischemic and non-ischemic origin. Therapeutic benefit was exemplified when given as an interventional treatment, i.e., after the onset of disease. Therapeutic benefit is also observed when given as a prophylactic treatment.

### SUMMARY OF THE INVENTION

The invention provides a method for treating heart failure in a patient. In one embodiment, the method comprises administering to a patient in need thereof an effective amount of Compound 1, or pharmaceutically-acceptable salts thereof.

In one embodiment of the invention, the heart failure to be treated results from hypertension, an ischemic heart disease, a non-ischemic heart disease, exposure to a cardiotoxic compound, myocarditis, Kawasaki's disease, Type I and Type II diabetes, thyroid disease, viral infection, gingivitis, drug abuse, alcohol abuse, pericarditis, atherosclerosis, vascular disease, hypertrophic cardiomyopathy, dilated cardiomyopathy, myocardial infarction, atrial fibrosis, left ventricular systolic dysfunction, left ventricular diastolic dysfunction, coronary bypass surgery, pacemaker implantation surgery, starvation, an eating disorder, muscular dystrophies, and a genetic defect.

In another embodiment of the invention, the heart failure to be treated is systolic heart failure, diastolic heart failure, heart failure with reduced ejection fraction (HF_{R}EF), heart failure with preserved ejection fraction (HF_{R}EF), acute heart failure, and chronic heart failure of ischemic and non-ischemic origin.

In another embodiment of the invention, Compound I of the invention is administered orally.

In yet another embodiment of the invention, the heart failure is HF_{R}EF, which results from myocardial infarction and the compound is administered either prior to or post the diagnosis of myocardial infarction in the patient.

In yet another embodiment of the invention, the compound is administrated 24 hours or 48 hours following myocardial infarction.

In yet another embodiment of the invention, the compound is administrated daily.

In yet another embodiment of the invention, the compound is administrated for at least a week, two weeks, three weeks, four weeks, five weeks or six weeks.

In yet another embodiment of the invention, administration of the compound improves left ventricular function.

In yet another embodiment of the invention, administration of the compound prevents progression of myocardial wall thinning.

In yet another embodiment of the invention, administration of the compound inhibits cardiomyocyte cell death.

In yet another embodiment of the invention, administration of the compound reduces infarct size.

In yet another embodiment of the invention, the heart failure is HFₚEF.

In yet another embodiment of the invention, administration of the compound improves myocardial wound healing.

In yet another embodiment of the invention, administration of the compound diminishes myocardial fibrosis.

In yet another embodiment, the present invention provides a combined preparation of Compound 1 of the present invention and additional therapeutic agent(s) for simultaneous, separate or sequential use in therapy.

In another embodiment, the invention provides Compound 1 and pharmaceutically-acceptable salts thereof for use in the prophylaxis and/or treatment of heart failure in a patient in need thereof.

In another embodiment, the invention provides use of Compound 1 for the prophylaxis and/or treatment of heart failure in a patient.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is a graph showing a dose-dependent reduction in LV anterior wall infarct size in mouse treated with Compound 1. Top panel: Infarct size is presented as percentage of total LV area. Bottom panel: Representative heart cross sections by histology depicting the degree of MI. Non-infarcted sham animals are shown for comparisons. Compound 1; LV, left ventricle; PO, orally, QD, once a day

FIG. 2 is a graph showing wall thickness of mice treated with vehicle and Compound 1. Transmural wall thickness of the MI was measured histologically for all groups. Dose-dependent preservation of infarct wall thickness with Compound 1 treatment is shown as % of sham. Vehicle indicates infarcted, no treatment; sham—non-infarcted, no treatment.

FIG. 3 is a graph showing LV chamber area of mice treated with vehicle and Compound 1. The area of the left ventricle chamber was measured for all groups from histological cross sections of the heart. Left ventricular dilatation caused by myocardial infarction increased chamber areas for all treatment groups relative to non-infarcted sham mice. Treatment with 0.3 mpk Compound 1 reduced LV chamber area relative to vehicle treatment.

FIG. 4 is a graph showing early infarct composition in mice treated with Compound 1 and vehicle. Top left: effect of Compound 1 on infarct area compared to vehicle. Top right; effect of Compound 1 on infarct collagen compared to vehicle. Bottom left: effect of Compound 1 on MMP-2 compared to vehicle. Bottom right: effect of Compound 1 on TIMP-4 compared to vehicle.

FIG. 5 is a graph showing dose-dependent preservation of infarct wall thickness in rats treated with Compound 1 and vehicle. Transmural wall thickness at the middle region of the MI measured histologically for all groups and is shown in the graph. Dose-dependent preservation of infarct wall thickness with Compound 1 treatment is shown as % of vehicle. Vehicle indicates infarcted, no drug treatment; sham—non-infarcted, no drug treatment

FIG. 6 is a graph showing improvement in left ventricle ejection fraction in rats treated with Compound 1 or vehicle. Echocardiography of rats occurred 6 weeks post treatment. Improvement in left ventricle ejection fraction was observed with Compound 1 vs. vehicle.

FIG. 7 is a graph showing dose-dependent improvement in left ventricular myocardial salvage in rats treated with Compound 1 or vehicle. Viable myocardium across the infarct wall at the middle region of the MI measured histologically for all groups. Preservation of viable myocardium with Compound 1 treatment is shown as % of vehicle. Vehicle indicates infarcted, no drug treatment.

FIG. 8 is a graph showing dose-dependent improvement in left ventricle ejection fraction in rats treated with Compound 1 or vehicle. Ejection fraction measurement as measured by pressure-volume conductance catheter 6 weeks post myocardial infarction.

FIG. 9 is a graph showing regression of cardiac fibrosis in mice by Compound 1. Fibrosis was stimulated with angiotensin II. Bottom: histology.

FIG. 10 is a graph showing reduction in cardiac fibrosis in mice by Compound 1. Fibrosis was stimulated with deoxycorticosterone acetate, a synthetic mimic of aldosterone.

### DETAILED DESCRIPTION OF THE INVENTION

### I. Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, in some instances ±5%, in some instances ±1%, and in some instances ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

As used herein, “heart failure” refers to an abnormality of cardiac structure or function leading to failure of the heart to deliver oxygen at a rate commensurate with the requirements of the metabolizing tissues, despite normal filling pressure. The underlying cause of heart failure can be due to systolic ventricular dysfunction or abnormalities of ventricular diastolic function.

As used herein “systolic ventricular dysfunction” or “systolic heart failure” refers to reduced contraction and emptying of the left ventricle. It occurs when the hearts left ventricle does not pump enough blood out into the body on each beat. Systolic heart failure is characterized by a reduced ejection fraction. Hence, systolic heart failure also is classified as heart failure with reduced ejection fraction.

As used herein, “diastolic ventricular dysfunction” or “diastolic heart failure” refers to abnormal heart relaxation and filling of the left ventricle. It occurs when the heart does not relax properly so that the heart is not able to fill with blood. Diastolic heart failure is characterized by a preserved ejection fraction, and hence also can be classified as heart failure with preserved ejection fraction.

As used herein, “reduced ejection fraction,” “heart failure with reduced ejection fraction” or “HF_{R}EF” refers to an ejection fraction of less than or equal to 50%, and generally less than or equal to 40%.

As used herein, “preserved ejection fraction,” or “heart failure with preserved ejection fraction” or “HF_{R}EF” refers to an ejection fraction of greater than or equal to 50%.

As used herein, “acute decompensated heart failure,” or “ADHF” refers to a worsening of the symptoms, typically shortness of breath (dyspnea), edema and fatigue, in a patient with existing heart disease. ADHF is a common and potentially serious cause of acute respiratory distress.

As used herein, “congestive heart failure” is meant impaired cardiac function that renders the heart unable to maintain the normal blood output at rest or with exercise, or to maintain a normal cardiac output in the setting of normal cardiac filling pressure. A left ventricular ejection fraction of about 40% or less is indicative of congestive heart failure (by way of comparison, an ejection fraction of about 60% percent is normal). Patients in congestive heart failure display well-known clinical symptoms and signs, such as tachypnea, pleural effusions, fatigue at rest or with exercise, contractile dysfunction, and edema.

As used herein, “ischemic heart disease” is meant any disorder resulting from an imbalance between the myocardial need for oxygen and the adequacy of the oxygen supply. Most cases of ischemic heart disease result from narrowing of the coronary arteries, as occurs in atherosclerosis or other vascular disorders.

As used herein, “left ventricular ejection fraction” or “LVEF” refers to the amount or percentage of blood pumped out of the total amount of blood in the left ventricle per beat. Thus, it is the percentage of blood pumped out of a filled left ventricle with each heartbeat. Generally, an LVEF >55% is normal, and lower than 50% is reduced. A skilled artisan is familiar with methods to assess or measure LVEF. Exemplary methods to measure EF include, but are not limited to, echocardiogram, cardiac catheterization, magnetic resonance imaging (MRI), computerized topography (CT) or nuclear medicine scan. EF can be measured as the stroke volume divided by end-diastolic volume.

As used herein, “diastole” refers to the cycle of heart pumping when the left ventricle fills with blood. The filling phase occurs when the heart muscle relaxes, allowing blood to enter and fill the left ventricle.

As used herein, “systole” refers to the cycle of heart pumping when the blood is forced out and the blood is emptied from the heart. The emptying phase occurs when the heart muscle contracts or squeezes to pump out or eject blood.

As used herein, “stroke volume” refers to the volume of blood pumped from one ventricle of the heart with each beat. Stroke volume is calculated as the end-diastolic volume minus the end-systolic volume.

As used herein, “end-diastolic volume” or “EDV” refers to the volume of blood in the ventricle at end load or filling in (i.e. diastole). Hence, it is the volume of blood just prior to the beat.

As used herein, “end-systolic volume” or “ESV” refers to the volume of blood in a ventricle at the end of contraction (i.e. systole) and the beginning of filling (i.e. diastole). Hence, it is the volume of the blood in the ventricle at the end of a beat. ESV can be used to clinically measure systolic function. Methods of assessing or measuring ESV are well known to a skilled artisan and include, but are not limited to, an electrocardiogram (the end of the T wave), echocardiography, MRI or CT.

As used herein, “ischemia-reperfusion injury,” is a type of ischemic event that is characterized biochemically by a depletion of oxygen during an ischemic event involving interrupted blood flow followed by reoxygenation and the concomitant generation of reactive oxygen species during reperfusion.

As used herein, “myocardial infarction” or “MI” is meant a process by which ischemic disease results in a region of the myocardium being replaced by scar tissue.

As used herein, “hypertension” is meant blood pressure that is considered by a medical professional (e.g., a physician or a nurse) to be higher than normal and to carry an increased risk for developing congestive heart failure.

As used herein, the term acute coronary syndrome, (ACS) refers to any group of symptoms attributed to obstruction of the coronary arteries. The most common symptom prompting diagnosis of ACS is chest pain, often radiating of the left arm or angle of the jaw, pressure-like in character, and associated with nausea and sweating.

As used herein, the term “signs and symptoms of heart disease” or “signs and symptoms of heart failure” refers to signs and symptoms associated with heart failure as recognized by simple observation or by standard clinical tests. This, when combined with an individual's age and family history of heart disease, can lead to diagnosis of heart disease or heart failure. Examples of signs of heart disease include, but are not limited to, dyspnea, chest pain (angina), palpitations, syncope, edema, cyanosis and fatigue. Among these are those that can be subject to quantitative analysis, such as palpitations, cyanosis and others. Other symptoms include discomfort or pressure in the chest, radiating discomfort to the back, jaw, throat or arm, fullness or ingestion, sweating, nausea, vomiting, dizziness, weakness or shortness of breath and/or rapid or irregular heartbeats It is within the level of a skilled artisan, such as a treating physician, to identify a sign or symptom of heart disease.

As used herein, “diseases and conditions associated with heart failure” refers to any condition associated with signs or symptoms of heart failure and that is confirmed by a diagnostic test of heart failure. Signs, symptoms and diagnostic tests for heart failure are well known to a skilled artisan. Symptoms of heart failure include, but are not limited to, breathlessness, ankle swelling or fatigue. Signs of heart failure include, but are not limited to, elevated jugular venous pressure, pulmonary crackles and displaced apex beat. Diagnostic tests for heart failure include, but are not limited to, abnormalities in the ability of heart to pump blood as determined by an electrocardiogram (EKG), an enlarged heart as determined by a chest x-ray, elevated levels of BNP in the blood, abnormal characteristics of heart size, shape, or blood flow as determined by an echocardiography (echo), abnormalities in pressure and blood flow in heart chambers as determined by cardiac catheterization, or abnormalities in blood flow as determined by coronary angiography. Exemplary of diseases and conditions associated with heart failure include, but are not limited to, ischemic heart disease (IHD; also called coronary heart disease), myocardial infarction, cardiomyopathy, high blood pressure, diseases of the heart valves, diseases of the pericardium, or arrhythmias.

As used herein, “at risk for congestive heart failure” is meant an individual who smokes, is obese (i.e., 20% or more over their ideal weight), has been or will be exposed to a cardiotoxic compound (such as an anthracycline antibiotic), or has (or had) high blood pressure, ischemic heart disease, a myocardial infarct, a genetic defect known to increase the risk of heart failure, a family history of heart failure, myocardial hypertrophy, hypertrophic cardiomyopathy, left ventricular systolic dysfunction, coronary bypass surgery, vascular disease, atherosclerosis, alcoholism, pericarditis, a viral infection, gingivitis, or an eating disorder (e.g., anorexia nervosa or bulimia), or is an alcoholic or cocaine addict.

As used herein, “decreasing progression of myocardial thinning” is meant maintaining hypertrophy of ventricular cardiomyocytes such that the thickness of the ventricular wall is maintained or increased.

As used herein, “patient” means a mammalian species, including humans, with a cardiovascular condition that is suitable for treatment as determined by practitioners in the field of cardiovascular diseases and conditions.

As used herein, “treating” or “treatment” cover a treatment of a disease-state in a mammal, particularly in a human, and include: (a) inhibiting a disease-state, i.e., arresting it development; and/or (b) relieving a disease-state, i.e., causing regression of a disease state; and/or (c) prophylaxis of a disease state. In particular, “treating” means that administration of Compound 1 slows or inhibits the progression of heart failure during the treatment, relative to the disease progression that would occur in the absence of treatment, in a statistically significant manner. Well known indicia such as left ventricular ejection fraction, exercise performance, and other clinical tests, as well as survival rates and hospitalization rates may be used to assess disease progression. Whether or not a treatment slows or inhibits disease progression in a statistically significant manner may be determined by methods that are well known in the art.

As used herein, “prophylaxis” is the protective treatment of a disease state to reduce and/or minimize the risk and/or reduction in the risk of recurrence of a disease state by administering to a patient a therapeutically effective amount of Compound 1, a tautomer, or a pharmaceutically acceptable salt thereof. Patients may be selected for prophylaxis therapy based on factors that are known to increase risk of suffering a clinical disease state compared to the general population. For prophylaxis treatment, conditions of the clinical disease state may or may not be presented yet. “Prophylaxis” treatment can be divided into (a) primary prophylaxis and (b) secondary prophylaxis. Primary prophylaxis is defined as treatment to reduce or minimize the risk of a disease state in a patient that has not yet presented with a clinical disease state, whereas secondary prophylaxis is defined as minimizing or reducing the risk of a recurrence or second occurrence of the same or similar clinical disease state.

As used herein, “risk reduction” covers therapies that lower the incidence of development of a clinical disease state. As such, primary and secondary prevention therapies are examples of risk reduction.

### II. Compound and Pharmaceutical Compositions

The present invention relates to Compound 1, compositions and methods for treating a patient having heart failure. The method comprises administering to a patient in need thereof, Compound 1, a tautomer, or pharmaceutically-acceptable salts thereof, at a therapeutically effective amount to treat heart failure.

Compound 1 is 1-((3S,4R)-4-(2,6-difluoro-4-methoxyphenyl)-2-oxopyrrolidin-3-yl)-3-phenylurea, which has the following structure:

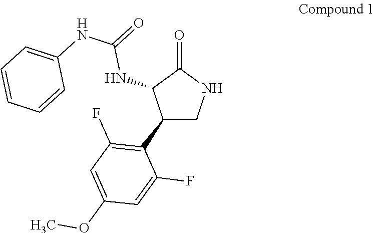 [SMILES: COc1cc(F)c([C@@H]2CNC(=O)[C@H]2NC(=O)Nc2ccccc2)c(F)c1]

The compound is disclosed in WO 2015/079692 A1 and its U.S. equivalent, U.S. Pub. No. 2017/0066718, which are herein incorporated by reference in their entities.

Without wishing to be bound by theory, the invention is partly based on the discovery that Compound 1 mediates chemotaxis and phagocytosis, reduces infarct size, improves myocardial cell survival, preserves ventricle wall thickness, enhances wound healing, and improves left ventricular ejection fraction after myocardial infarction. In addition, the invention is based partly on the discovery that the compound can also reduce cardiac fibrosis of non-ischemic origin.

It will be understood that treatment or prophylaxis of heart failure may involve treatment or prophylaxis of a cardiovascular event as well. Treatment or prophylaxis as referred to herein may refer to treatment or prophylaxis of certain negative symptoms or conditions associated with or arising as a result of a cardiovascular event. By way of example, treatment or prophylaxis may involve reducing or preventing negative changes in fractional shortening, heart weight, lung weight, myocyte cross sectional area, pressure overload induced cardiac fibrosis, stress induced cellular senescence, and/or cardiac hypertrophy properties, or any combination thereof, associated with or arising as a result of a cardiovascular event. Treatment may be administered in preparation for or in response to a cardiovascular event to alleviate negative effects. Prevention may involve a pro-active or prophylactic type of treatment to prevent the cardiovascular event or to reduce the onset of negative effects of a cardiovascular event.

In one embodiment, the present invention provides the use of Compound 1 or a pharmaceutically acceptable salt thereof for the preparation of a pharmaceutical composition for the treatment or prophylaxis of heart failure, for example, heart failure results from hypertension, an ischemic heart disease, a non-ischemic heart disease, exposure to a cardiotoxic compound, myocarditis, Kawasaki's disease, Type I and Type II diabetes, thyroid disease, viral infection, gingivitis, drug abuse, alcohol abuse, pericarditis, atherosclerosis, vascular disease, hypertrophic cardiomyopathy, dilated cardiomyopathy, myocardial infarction, atrial fibrosis, left ventricular systolic dysfunction, left ventricular diastolic dysfunction, coronary bypass surgery, pacemaker implantation surgery, starvation, an eating disorder, muscular dystrophies, and a genetic defect. Preferably, the heart failure to be treated is diastolic heart failure, heart failure with reduced ejection fraction (HF_{R}EF), heart failure with preserved ejection fraction (HF_{P}EF), acute heart failure, and chronic heart failure of ischemic and non-ischemic origin.

In one embodiment, the present invention provides the use of Compound 1 to treat systolic and/or diastolic dysfunction, wherein Compound 1 is administered in a therapeutically effective amount to increase the ability of the cardiac muscle cells to contract and relax thereby increasing the filling and emptying of both the right and left ventricles, preferably, the left ventricle.

In another embodiment, the present invention provides the use of Compound 1 to treat heart failure wherein Compound 1 is administered in a therapeutically effective amount to increase ejection fraction in the left ventricle.

In still another embodiment, the present invention provides the use of Compound 1 to treat heart failure wherein Compound 1 is administered in a therapeutically effective amount to reduce hypertrophy in heart tissue.

In still another embodiment, the present invention provides the use of Compound 1 to treat heart failure wherein Compound 1 is administered in a therapeutically effective amount to prevent progression of myocardial wall thinning.

In yet another embodiment, the present invention provides the use of Compound 1 to treat heart failure wherein Compound 1 is administered in a therapeutically effective amount to inhibit cardiomyocyte cell death.

In yet another embodiment, the present invention provides the use of Compound 1 to treat heart failure wherein Compound 1 is administered in a therapeutically effective amount to reduce infarct size.

In still another embodiment, the present invention provides the use of Compound 1 to treat heart failure wherein Compound 1 is administered in a therapeutically effective amount to reduce fibrosis in heart tissue.

The invention includes all pharmaceutically acceptable salt forms of Compound 1. Pharmaceutically acceptable salts are those in which the counter ions do not contribute significantly to the physiological activity or toxicity of the compounds and as such function as pharmacological equivalents. These salts can be made according to common organic techniques employing commercially available reagents. Some anionic salt forms include acetate, acistrate, besylate, bromide, chloride, citrate, fumarate, glucouronate, hydrobromide, hydrochloride, hydroiodide, iodide, lactate, maleate, mesylate, nitrate, pamoate, phosphate, succinate, sulfate, tartrate, tosylate, and xinofoate. Some cationic salt forms include ammonium, aluminum, benzathine, bismuth, calcium, choline, diethylamine, diethanolamine, lithium, magnesium, meglumine, 4-phenylcyclohexylamine, piperazine, potassium, sodium, tromethamine, and zinc.

The present invention provides pharmaceutical compositions comprised of a therapeutically effective amount of Compound 1 and a pharmaceutically acceptable carrier. The term “pharmaceutical composition” means a composition comprising a compound of the invention in combination with at least one additional pharmaceutically acceptable carrier. A “pharmaceutically acceptable carrier” refers to media generally accepted in the art for the delivery of biologically active agents to animals, in particular, mammals, including, i.e., adjuvant, excipient or vehicle, such as diluents, preserving agents, fillers, flow regulating agents, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms. These agents are included in the formulation for a variety of reasons, e.g., stabilization of the active agent, binders, etc. Pharmaceutically acceptable carriers include both aqueous and non-aqueous liquid media, as well as a variety of solid and semi-solid dosage forms. Descriptions of suitable pharmaceutically acceptable carriers, and factors involved in their selection, are found in a variety of readily available sources such as, for example, Allen, Jr., L. V. et al., Remington: The Science and Practice of Pharmacy (2 Volumes), 22nd Edition, Pharmaceutical Press (2012). A “therapeutically effective amount” means the amount of agent required to provide a meaningful patient benefit as understood by practitioners in the field of cardiovascular diseases and conditions.

Compositions encompass all common solid and liquid forms including capsules, tablets, losenges, and powders as well as liquid suspensions, syrups, elixers, and solutions. Compositions are made using common formulation techniques, and conventional excipients (such as binding and wetting agents) and vehicles (such as water and alcohols) are generally used for compositions. See, for example, Remington's Pharmaceutical Sciences, 22^{nd} edition, Mack Publishing Company, Easton, Pa. (2013).

Pharmaceutically acceptable carriers are formulated according to a number of factors well within the purview of those of ordinary skill in the art. These include, without limitation: the type and nature of the active agent being formulated; the subject to which the agent-containing composition is to be administered; the intended route of administration of the composition; and the therapeutic indication being targeted.

Pharmaceutical compositions suitable for administration may contain from about 0.1 milligram to about 2000 milligrams of active ingredient per dosage unit. In these pharmaceutical compositions the active ingredient will ordinarily be present in an amount of about 0.1-95% by weight based on the total weight of the composition.

The concentration of Compound 1 in the formulations is effective for delivery of an amount, upon administration, that is effective for the intended treatment. Those of skill in the art readily can formulate a composition for administration in accord with the methods herein. For example, to formulate a composition, the weight fraction of a compound or mixture thereof is dissolved, suspended, dispersed, or otherwise mixed in a selected vehicle at an effective concentration such that heart failure is improved.

The precise amount or dose of the therapeutic agent administered depends on the route of administration, and other considerations, the weight and general state of the subject and the subject and the status of the heart failure. Local administration of the therapeutic agent will typically require a smaller dosage than any mode of systemic administration, although the local concentration of the therapeutic agent can, in some cases, be higher following local administration than can be achieved with safety upon systemic administration. For the methods herein, Compound 1 generally is administered orally.

### III. Administration and Dosage Regimen

In the methods herein, Compound 1 can be administered to a subject for treating heart failure, and in particular any disease or condition associated with heart failure. Compound 1 is intended for use either as a stand-alone agent or in combination with other therapeutic methods including mechanical devices or pharmacological medications, which can lower blood pressure or otherwise treat heart failure and/or heart dysfunction.

Active agents, for example, Compound 1, are included in an amount sufficient that exert a therapeutically useful effect in the absence of undesirable side effects on the patient treated. The amount of Compound 1 to be administered for the treatment heart failure, for example for treating a patient with HF_{P}EF, can be determined by standard clinical techniques. In addition, in vitro assays and animal models can be employed to help identify optimal dosage ranges. The precise dosage, which can be determined empirically, can depend on the particular composition, the route of administration, the type of disease to be treated and the seriousness of the disease.

Compound 1 is administered in a single dose or multiple doses at intervals as described herein to produce a long lasting effect on cardiac function without any toxicity. In some examples, methods of treatment with Compound 1 requires a longer duration of action in order to effect a sustained therapeutic effect. This is particularly true in treatment of chronic heart failure. Thus, Compound 1 described herein can be used to deliver longer lasting therapies for cardiac disorders.

If necessary, a particular dosage and duration and treatment protocol can be empirically determined or extrapolated. For example, Compound 1, if necessary, can be used as a starting point to determine appropriate dosages for a particular subject and condition. The duration of treatment and the interval between injections will vary with the severity of the disease or condition and the response of the subject to the treatment, and can be adjusted accordingly. Factors such as the level of activity and half-life of the compound, can be taken into account when making dosage determinations. Particular dosages and regimens can be empirically determined by one of skill in the art.

The dosage regimen for Compound 1 will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired.

Treatment of diseases and conditions with Compound 1 can be effected by any suitable route of administration using suitable formulations as described herein including, but not limited to, injection, pulmonary, oral and transdermal administration. Treatment typically is effected by oral administration.

If necessary, a particular dosage and duration and treatment protocol can be empirically determined or extrapolated. Dosages for FPR2 agonists previously administered to human subjects and used in clinical trials can be used as guidance for determining dosages for Compound 1. Dosages for Compound 1 can also be determined or extrapolated from relevant animal studies. Factors such as the level of activity and half-life of Compound 1 can be used in making such determinations. Particular dosages and regimens can be empirically determined based on a variety of factors. Such factors include body weight of the individual, general health, age, the activity of the specific compound employed, sex, diet, time of administration, rate of excretion, drug combination, the severity and course of the disease, and the patient's disposition to the disease and the judgment of the treating physician. The active ingredient, Compound 1 typically is combined with a pharmaceutically effective carrier. The amount of active ingredient that can be combined with the carrier materials to produce a single dosage form or multi-dosage form can vary depending upon the host treated and the particular mode of administration.

In particular examples, Compound 1 is formulated for administration to a patient at a dosage of about 0.01 to 100 mg/kg patient body weight, such as 0.05 to 50 mg/kg patient body weight, for example 0.1 mg/kg to 10 mg/kg, 0.1 to 20 mg/kg, 0.1 mg/kg to 30 mg/kg, 0.1 mg/kg to 40 mg/kg.

In a patient with heart failure the goal is to administer the dose in the smallest volume possible. Typically, the volume to be administered is not greater than 4.0 mL/kg of a subject. For example, the volume in which the dose is administered to a subject can be 0.4 mL/kg to 4.0 mL/kg. For example, a composition with a concentration of 22.5% (i.e. 225 mg/mL) that is administered to a 100 kg subject at a dose of 100 mg/kg would require a volume of about 44 mL or about 0.4 mL/kg to achieve that dose.

By way of general guidance, the daily oral dosage of each active ingredient, when used for the indicated effects, will range between about 0.001 to about 5000 mg per day, preferably between about 0.01 to about 1000 mg per day, and most preferably between about 0.1 to about 250 mg per day. Compound 1 may be administered in a single daily dose, or the total daily dosage may be administered in divided doses of two, three, or four times daily.

The length of time of the cycle of administration can be empirically determined, and is dependent on the disease to be treated, the severity of the disease, the particular patient, and other considerations within the level of skill of the treating physician. The length of time of treatment with Compound 1 can be one day, one week, two weeks, one months, several months, one year, several years or more. If disease symptoms persist in the absence of discontinued treatment, treatment can be continued for an additional length of time. Over the course of treatment, evidence of disease and/or treatment-related toxicity or side effects can be monitored.

In addition, the cycle of administration can be tailored to add periods of discontinued treatment in order to provide a rest period from exposure to the treatment. The length of time for the discontinuation of treatment can be for a predetermined time or can be empirically determined depending on how the patient is responding or depending on observed side effects. For example, the treatment can be discontinued for one week, two weeks, one month or several months.

Effective treatment can be exhibited by an increase of ejection fraction, increase in diastolic and/or systolic function, improvement in hemodynamics, reduction in inflammatory cytokine levels and neurohormone levels, reduction in markers of inflammation, reduction in injury markers, inhibition of platelet aggregation, improvement in endothelial function, reductions in arrhythmias, and improvement in heart rate variability, improvement in QRS dispersion and QTC prolongation, and improved immune responsiveness, all of which can be tested by skilled artisans with known and available testing regimes.

For treatments involving acute heart failure, dosing will typically start when the patient is admitted to the hospital, but it can be started any time during hospitalization to meet the subject's needs. More generally, the dosing can start during the first 72 hours of hospitalization. For example, dosing can start 24 or 48 hours after the diagnosis of myocardial infarction, the occurrence of a vascular injury or surgical operation. For chronic heart failure, dosing generally is provided based on the needs of the subject, since such subjects generally do not undergo hospitalization. For example, treatment can be started 1 and 7 days after the diagnosis of heart failure.

Compound 1 can be administered daily, at 1-2 week intervals, 2-3 week intervals, 3-4 week intervals, 4-5 week intervals or 5-6 week intervals at variable doses. In other regimens, the interval between doses can be increased over time, whereby the second dose is administered 1-6 days, 1-2, or 2-3 weeks after the first dose, the third dose administered 1-6 days, 1-2, or 2-3 weeks after the second dose and the fourth and any subsequent dose is administered 1-6 days, 1-2, or 2-3 weeks following the previous dose. As performance of the heart improves, as assessed by standard parameters, such as those described herein, the dose can be titrated downward depending on the needs of the patient.

### IV. Combinations

Compound 1 of the present invention can be used alone, or in combination with one or more other therapeutic agent(s), e.g., agents used in treatment of heart failure or other pharmaceutically active material. By “administered in combination” or “combination therapy” it is meant that the compound of the present invention and one or more additional therapeutic agents are administered concurrently to the mammal being treated. When administered in combination, each component may be administered at the same time or sequentially in any order at different points in time. Thus, each component may be administered separately but sufficiently closely in time so as to provide the desired therapeutic effect.

It is within the level of a skilled artisan to choose a further additional treatment to administer in conjunction with a therapeutic regime employing Compound 1. Such a decision will depend on the particular disease or condition being treated, the particular subject being treated, the age of the subject, the severity of the disease or condition and other factors.

Compound 1 may be employed in combination with other FPR2 agonists or one or more other suitable therapeutic agents useful in the treatment of the aforementioned disorders including: other agents for treating heart failure, inotropic agents, anti-hypertensive agents, anti-atherosclerotic agents, anti-hyperlipidemic agents, plasma HDL-raising agents, anti-hypercholesterolemic agents, anti-dyslipidemic agents, anti-diabetic agents, anti-hyperglycemic agents, anti-hyperinsulinemic agents, anti-thrombotic agents, anti-retinopathic agents, anti-neuropathic agents, anti-nephropathic agents, anti-ischemic agents, anti-obesity agents, anti-hyperlipidemic agents, anti-hypertriglyceridemic agents, anti-hypercholesterolemic agents, anti-restenotic agents, anti-pancreatic agents, lipid lowering agents, anorectic agents, memory enhancing agents, anti-dementia agents, cognition promoting agents, appetite suppressants, and agents for treating peripheral arterial disease.

In particular, Compound 1 of the present invention may be employed in combination with additional therapeutic agent(s) selected from one or more, preferably one to three, of the following therapeutic agents in treating heart failure and coronary artery disease: loop diuretics, Angiotensin converting enzyme (ACE) inhibitors, Angiotensin II receptor blockers (ARBs), angiotensin receptor-neprilysin inhibitors (ARNI), β-blockers, mineralocorticoid receptor antagonists, nitroxyl donors, RXFP1 agonists, APJ agonists and cardiotonic agents, renin inhibitors, calcium channel blockers, angiotensin II receptor antagonists, nitrates, digitalis compounds, and β-receptor agonists, cholesterol biosynthesis inhibitors (such as HMG CoA reductase inhibitors), Sodium-glucose co-transporter 2 inhibitors and LXR agonist. These agents include, but are not limited to furosemide, bumetanide, torsemide, sacubittrial-valsartan, thiazide diruetics, captopril, enalapril, lisinopril carvedilol, metopolol, bisoprolol, serelaxin, spironolactone, eplerenone, ivabradine, candesartan, eprosartan, irbestarain, losartan, olmesartan, telmisartan, and valsartan, probucol, raloxifene, nicotinic acid, niacinamide, cholesterol absorption inhibitors, bile acid sequestrants (such as anion exchange resins, or quaternary amines (e.g., cholestyramine or colestipol), low density lipoprotein receptor inducers, clofibrate, fenofibrate, benzofibrate, cipofibrate, gemfibrizol, vitamin B₆, vitamin B₁₂, anti-oxidant vitamins, anti-diabetes agents, platelet aggregation inhibitors, fibrinogen receptor antagonists, aspirin and fibric acid derivatives.

The above other therapeutic agents, when employed in combination with Compound 1 may be used, for example, in those amounts indicated in the Physicians' Desk Reference, as in the patents set out above, or as otherwise determined by one of ordinary skill in the art.

Particularly when provided as a single dosage unit, the potential exists for a chemical interaction between the combined active ingredients. For this reason, when the compound of the present invention and a second therapeutic agent are combined in a single dosage unit they are formulated such that although the active ingredients are combined in a single dosage unit, the physical contact between the active ingredients is minimized (that is, reduced). For example, one active ingredient may be enteric coated. By enteric coating one of the active ingredients, it is possible not only to minimize the contact between the combined active ingredients but also to control the release of one of these components in the gastrointestinal tract such that one of these components is not released in the stomach but rather is released in the intestines. One of the active ingredients may also be coated with a material that affects a sustained-release throughout the gastrointestinal tract and also serves to minimize physical contact between the combined active ingredients. Furthermore, the sustained-released component can be additionally enteric coated such that the release of this component occurs only in the intestine. Still another approach would involve the formulation of a combination product in which the one component is coated with a sustained and/or enteric release polymer, and the other component is also coated with a polymer such as a low viscosity grade of hydroxypropyl methylcellulose (HPMC) or other appropriate materials as known in the art, in order to further separate the active components. The polymer coating serves to form an additional barrier to interaction with the other component.

These as well as other ways of minimizing contact between the components of combination products of the present invention, whether administered in a single dosage form or administered in separate forms but at the same time by the same manner, will be readily apparent to those skilled in the art, once armed with the present disclosure.

The present invention also encompasses an article of manufacture. As used herein, article of manufacture is intended to include, but not be limited to, kits and packages. The article of manufacture of the present invention, comprises: (a) a first container; (b) a pharmaceutical composition located within the first container, wherein the composition, comprises a first therapeutic agent, comprising a compound of the present invention or a pharmaceutically acceptable salt form thereof; and, (c) a package insert stating that the pharmaceutical composition can be used for the treatment and/or prophylaxis of heart failure. In another embodiment, the package insert states that the pharmaceutical composition can be used in combination (as defined previously) with a second therapeutic agent for the treatment and/or prophylaxis of heart failure. The article of manufacture can further comprise: (d) a second container, wherein components (a) and (b) are located within the second container and component (c) is located within or outside of the second container. Located within the first and second containers means that the respective container holds the item within its boundaries.

The first container is a receptacle used to hold a pharmaceutical composition. This container can be for manufacturing, storing, shipping, and/or individual/bulk selling. First container is intended to cover a bottle, jar, vial, flask, syringe, tube (e.g., for a cream preparation), or any other container used to manufacture, hold, store, or distribute a pharmaceutical product.

The second container is one used to hold the first container and, optionally, the package insert. Examples of the second container include, but are not limited to, boxes (e.g., cardboard or plastic), crates, cartons, bags (e.g., paper or plastic bags), pouches, and sacks. The package insert can be physically attached to the outside of the first container via tape, glue, staple, or another method of attachment, or it can rest inside the second container without any physical means of attachment to the first container. Alternatively, the package insert is located on the outside of the second container. When located on the outside of the second container, it is preferable that the package insert is physically attached via tape, glue, staple, or another method of attachment. Alternatively, it can be adjacent to or touching the outside of the second container without being physically attached.

The package insert is a label, tag, marker, etc. that recites information relating to the pharmaceutical composition located within the first container. The information recited will usually be determined by the regulatory agency governing the area in which the article of manufacture is to be sold (e.g., the United States Food and Drug Administration). Preferably, the package insert specifically recites the indications for which the pharmaceutical composition has been approved. The package insert may be made of any material on which a person can read information contained therein or thereon. Preferably, the package insert is a printable material (e.g., paper, plastic, cardboard, foil, adhesive-backed paper or plastic, etc.) on which the desired information has been formed (e.g., printed or applied).

Compound 1 is also useful as a standard or reference compound, for example as a quality standard or control, in tests or assays involving the FPR2 receptor. Such a compound may be provided in a commercial kit, for example, for use in pharmaceutical research involving FPR2 or anti-heart failure activity. For example, Compound 1 could be used as a reference in an assay to compare its known activity to a compound with an unknown activity. This would ensure the experimenter that the assay was being performed properly and provide a basis for comparison, especially if the test compound was a derivative of the reference compound. When developing new assays or protocols, Compound 1 could be used to test their effectiveness. Compound 1 of the present invention may also be used in diagnostic assays involving heart failure.

Other features of the invention will become apparent in the course of the following descriptions of exemplary embodiments. The examples are offered as illustrative, as a partial scope and particular embodiments of the invention and are not meant to be limiting of the scope of the invention.

### V. Examples

### Example 1

### FPR2 and FPR1 cAMP Assays

A mixture of forskolin (5 μM final for FPR2 or 10 μM final for FPR1) and IBMX (200 μM final) were added to 384-well Proxiplates (Perkin-Elmer) pre-dotted with test compounds in DMSO (1% final) at final concentrations in the range of 0.1 nM to 10 μM. CHO overexpressing human FPR1 or human FPR2 receptors were cultured in F-12 (Ham's) medium supplemented with 10% qualified FBS, 250 μg/ml zeocin and 300 μg/ml hygromycin (Life Technologies). Reactions were initiated by adding 2,000 human FPR2 cells per well or 4,000 human FPR1 cells per well in Dulbecco's PBS (with calcium and magnesium) (Life Technologies) supplemented with 0.1% BSA (Perkin-Elmer). The reaction mixtures were incubated for 30 min at room temperature. The level of intracellular cAMP was determined using the HTRF HiRange cAMP assay reagent kit (Cisbio) according to manufacturer's instruction. Solutions of cryptate conjugated anti-cAMP and d2 flurorophore-labelled cAMP were made in a supplied lysis buffer separately. Upon completion of the reaction, the cells were lysed with equal volume of the d2-cAMP solution and anti-cAMP solution. After a 1 h room temperature incubation, time-resolved fluorescence intensity was measured using the Envision (Perkin-Elmer) at 400 nm excitation and dual emission at 590 nm and 665 nm. A calibration curve was constructed with an external cAMP standard at concentrations ranging from 1 μM to 0.1 pM by plotting the fluorescent intensity ratio from 665 nm emission to the intensity from the 590 nm emission against cAMP concentrations. The potency and activity of Compound 1 to inhibit cAMP production was then determined by fitting to a 4-parametric logistic equation from a plot of cAMP level versus compound concentrations.

### β-Arrestin Recruitment Assay

PathHunter® β-Arrestin GPCR cell lines are engineered to co-express the ProLink™ (PK) tagged FPR2 and the Enzyme Acceptor (EA) tagged β-Arrestin. Activation of the GPCR-PK induces β-Arrestin-EA recruitment, forcing complementation of the two β-galactosidase enzyme fragments (EA and PK). The resulting functional enzyme hydrolyzes substrate to generate a chemiluminescent signal Human FPR2 β-arrestin PathHunter cells (DiscoveRx) were cultured in F-12 (Ham's) medium supplemented with 10% heat inactivated FBS, 800 μg/mL Genetcin, 300 μg/mL Hygromycin B, 1× L-Glutamine (Life Technologies). One day prior to assay, the cells were harvested into a plating medium (DMEM/F-12 supplemented with 1% heat inactivated FBS) and seeded into 96 well assay plate (Corning Costar 3603) at 30,000 cells/well (100 μL) for overnight incubation. Test compounds were solubilized and serially diluted in DMSO to final assay concentrations in the range of 0.2 nM to 10 μM. Reactions were initiated by transferring the test compounds to the cell plates. The reaction mixtures were incubated at 37° C. for 90 min. Subsequently, detection reagents containing Cell Assay Buffer, Emerald II and Galacton-Star (19:5:1) were prepared per manufacturer's instructions and added to the cells (10 μL). The mixtures were incubated for 1 to 2 h at room temperature in the dark. Chemiluminescence signal was measured on the Envision (Perkin-Elmer). Data were fitted to a 4-parametric logistic equation from a plot normalized signal versus compound concentrations. The potency of the test compound is reported as the EC₅₀ value derived from the fitted curve.

### HL-60 Cell Culture and Differentiation

The HL-60 cell line (ATCC, CCL-240, lot 60398411) was maintained in IMDM (Life Tech, cat 12440-053) medium supplemented with 20% fetal bovine serum, 50 U/ml penicillin, and 50 μg/ml streptomycin at 37° with 5% CO₂. Cells were differentiated into the granulocyte lineage with DMSO; 2.5×10⁵ cells/ml were incubated with 1.25% DMSO for 5 days.

### Neutrophil and HL-60 Cell Migration Assay Agonist Mode

After 5 day differentiation, cells were resuspended in phenol free RPMI (Invitrogen, cat 11835) with 0.2% fatty acid free BSA at a concentration of 3×10⁷ cells/ml. The dHL-60 cells (10⁵ in 100 μl) were added to the upper chamber of each HTS transwell-96 well plate (Corning #3387). Migration was induced by placing Compound 1 in the bottom chamber and the dHL60 cells in the top chamber of the transwell plate. Cells were allowed to migrate for 120 min across the 5 micron filters at 37° C. with 5% CO₂. Following migration, neutrophils or dHL-60 cells remaining in the transwell lower chamber (migrated fraction) were quantitated using the cell-titer-glo luminescence cell viability assay (Promega, G7571).

### Neutrophil and HL-60 Cell Migration Assay Antagonist Mode

After 5 day differentiation, the cells were resuspended in phenol free RPMI (Invitrogen, cat 11835) with 0.2% fatty acid free BSA at a concentration of 3×10⁷ cells/ml. The dHL-60 cells (10⁵ in 100 μl) were pre-incubated for 15 minutes with varying concentrations of the chemoattractant at 37° with 5% CO₂. Then 0.8 μM of the recombinant serum amyloid A1 peptide (rSAA1, PeproTech, Cat #300-53) was added to the bottom chamber of each HTS transwell-96 well plate (Corning #3387). Migration was induced by placing chemoattractant and the dHL60 cells mixture in the top chamber of the transwell plate. Cells were allowed to migrate for 120 min across the 5 micron filters at 37° C. with 5% CO₂. Following migration, neutrophils or dHL-60 cells remaining in the transwell lower chamber (migrated fraction) were quantitated using the cell-titer-glo luminescence cell viability assay (Promega, G7571).

Enhancement of phagocytosis Macrophages were elicited to the peritoneum of five C57BL6 mice by peritoneal injection of 1 mL of 1% Biogel in PBS (−/−) 4 days prior to harvest. Peritoneal exudates are harvested, combined and then filtered to remove Biogel beads. First, through a 70 um cell strainer followed by successively filtering through two 40 um cell strainers. The exudate is diluted with 1×PBS (−/−) to 50 mL and centrifuged at 300×g for 10 minutes at 4° C. The cell pellet is gently resuspended in 20-30 mL 1×PBS(+/+) and cells are counted using the Nexelcom Cellometer counter. Cell concentration is adjusted to 1,250,000 cells/mL in 1×PBS (+/+). 100 μL (125 k) cells are placed into each well of a 96-well Costar 3904 plate. The plates are centrifuged at 150×g for 30 seconds to promote adherence. After 90 minutes incubation at 37° C./5% CO₂, non-adherent cells are aspirated and attached macrophages (˜50K) are washed once with 150 μL 1×PBS (−/−) and then incubated overnight at 37° C./5% CO₂, in 135 μL pre-warmed serum-free Macrophage SFM/1X Pen-Strep media. The following day, 15 uL of freshly prepared 10× compound 1 in serum-free Macrophage SFM media is added to each well, mixed and incubated for 15 minutes at 37° C./5% CO₂. Phagocytosis is initiated by the addition of a 10-fold excess (4 μL of 125K/μL) of opsonized FITC Zymosan particles (Life Technologies). Phagocytosis is allowed to proceed for 45 minutes at 37° C./5% CO₂. Wells are aspirated, phagocytosis is arrested with 150 μL of ice-cold 1×PBS (−/−)/2 mM EDTA and aspirated again. Fluorescence signal from non-ingested Zymosan particles is quenched with 150 uL ice-cold 1:15 diluted Trypan Blue solution for 2 minutes and then aspirated to remove. Lastly, the plate is read on a SpectraMAX Gemini EM fluorescence plate reader in 150 ul of 1:50 diluted Trypan Blue. Plate Reader Settings=Bottom Read: Excitation 493 nm: Emission 525 nm: Cutoff 515 nm: Automix Off: Calibrate On: PMT=Auto: Column Priority: Reads/Well=20.

### Example 2

### In Vitro FPR2 and FPR1 Activity for Compound 1

Agonist activity of Compound 1 for FPR2 and the closely related isoform, FPR1, was characterized in a panel of in vitro functional assays utilizing FPR-expressing cell lines. In CHO-A12 cell lines, one over-expressing human FPR2 (hFPR2) and the other overexpressing human FPR1 (hFPR1); Compound 1 was a potent activator of hFPR2 Gi coupling (EC₅₀=2.8 nM), resulting in lowering of cyclic adenosine monophosphate cAMP levels through adenylcyclase inhibition. Compound 1 was a significantly less potent activator of the related hFPR1 receptor (EC₅₀=1250 nM). In the CHO-A12 cell line over-expressing mouse ortholog, mFPR2, Compound 1 was a very potent activator of mFPR2 (EC₅₀=0.6 nM). Similar to results observed with the human FPR2 cell lines, Compound 1 was also selective for mFPR2 and rat FPR2, with functional potencies of approximately 384 nM against the mFPR1 receptor and 720 nM for rat FPR1. In CHO cell lines expressing human, mouse, or rat FPR2 or FPR1, Compound 1 showed the following selectivity: 446-fold for human FPR2 vs. FPR1; 640-fold for mouse FPR2 vs. FPR1; 480-fold for rat FPR2 vs. FPR1. Results are summarized in Table 1.

TABLE 1
Characterization of Compound 1 agonist activity in vitro

| Assay | Cell line | EC₅₀, nM |
|---|---|---|
| cAMP Inhibition | Human FPR2/CHO-A12 | 2.8 |
| | Human FPR1/CHO-A12 | 1250 |
| | Mouse FPR2/CHO-A12 | 0.6 |
| | Mouse FPR1/CHO-A12 | 384 |
| | Rat FPR2 | 1.5 |
| | Rat FPR1 | 720 |

Abbreviations:
cAMP, cyclic adenosine monophosphate;
CHO, Chinese hamster ovary;
FPR, formyl peptide receptor

### Example 3

### Effect of Compound 1 on Chemotaxis and Phagocytosis

Using the human promyelocytic leukemia HL-60 cell line and primary mouse peritoneal macrophages, the potencies of Compound 1 on chemotaxis and enhancement of phagocytosis were investigated.

Compound 1 antagonized chemotaxis of HL-60 cell line stimulated by pro-inflammatory ligand SAA with an IC₅₀ of 57 nM. In mouse peritoneal macrophages elicited with 1% Biogel, Compound 1 enhanced phagocytosis of fluorescently labeled zymosan with an EC₅₀ of 2 nM Results are summarized in Table 2.

TABLE 2
Characterization of chemotactic and phagocytic activity of
Compound 1 in vitro

| Assay | Cell line | Activity |
|---|---|---|
| Chemotaxis | HL-60 | IC₅₀ = 57 nM |
| Zymozan stimulated | Mouse peritoneal macrophages | EC₅₀ = 2 nM |
| phagocytosis | | |

### Example 4

### Animal Models

Permanent coronary artery occlusion was carried out in mice using a ligature placed around the left anterior descending artery to induce myocardial infarction. Treatment with orally-administered Compound 1 (0.3 and 3 mg/kg; QD) or dosing solution without compound (QD, referred to as vehicle) was initiated 24 hours following myocardial infarction. Mice subjected to thoracotomy but not infarcted were included as surgical “sham” controls. Mice were evaluated 28 days following myocardial infarction to assess structure/function relationships. Hearts were removed from mice for histological processing to measure left ventricular dimensions, infarct areas and infarct collagen composition.

In a second model of myocardial infarction, permanent coronary artery occlusion was carried out in rats using a ligature placed around the left anterior descending artery to induce myocardial infarction. Treatment with orally-administered Compound 1 (0.01, 0.1, 1, and 10 mg/kg, QD) or dosing solution without compound (QD, referred to as vehicle) was initiated 48 hours following myocardial infarction. Rats subjected to thoracotomy but not infarcted were included as surgical “sham” controls. Rats were evaluated 6 weeks following myocardial infarction to assess structure/function relationships. Hearts were removed from rats for histological processing to measure infarct wall thickness and collagen composition. Echocardiography was carried out to evaluate cardiac function.

In a third model of myocardial infarction, transient coronary artery occlusion followed by reperfusion was carried out in rats to induce myocardial infarction and to assess salvage of myocardium within the area at risk of myocardial infarction. Treatment with orally-administered Compound 1 (0.01, 0.1, 1, and 10 mg/kg, QD) or dosing solution without compound (QD, referred to as vehicle) was initiated 48 hours following myocardial infarction. Rats subjected to thoracotomy but not infarcted were included as surgical “sham” controls. Rats were evaluated 6 weeks following myocardial infarction to assess structure/function relationships. Hearts were removed from rats for histological processing to the extent of myocardial salvage with Compound 1 treatment. The pressure-volume conductance catheter technique was used to evaluate cardiac function.

To assess myocardial fibrosis in the setting of non-ischemic heart disease with hypertension, mice were challenged with angiotensin II to stimulate cardiac hypertrophy and left ventricular fibrosis. Mice were administered angiotensin II using subcutaneously implanted osmotic mini-pumps (˜2 mg/kg/day). A separate group of mice were implanted with subcutaneous pumps containing saline (surgical “sham” group); these mice served as control for pump implantation surgery. Mice were treated with Compound 1 (3 mg/kg, QD) or dosing solution without compound (QD, referred to as vehicle) starting 7 days following pump implantation. In this model, cardiac fibrosis development reaches its peak and without treatment, will remain fibrotic at this high level until the angII is depleted (˜4 weeks post implantation). Interventional treatment with Compound 1 to regress cardiac fibrosis was given to mice for 3 weeks. At the end of treatment phase, hearts were removed from animals and evaluated for collagen levels/fibrosis by cross-sectional histology of the hearts.

In a second model of non-ischemic heart disease with hypertension, mice were subjected to uni-nephrectomy and challenged with deoxycorticosterone acetate (DOCA) and saline drinking water to stimulate cardiac hypertrophy and left ventricular fibrosis. Mice were administered DOCA using subcutaneously implanted pellets (50 mg). A separate group of mice were subjected to surgery via creation of subcutaneous pocket (surgical “sham” group); these mice served as control for pellet implantation surgery. Mice were treated with Compound 1 (1 mg/kg, QD) or dosing solution (QD, referred to as vehicle) 24 hours after surgery. Treatment with Compound 1 was given to mice for 3 weeks. At the end of treatment phase, hearts were removed from animals and evaluated for collagen levels/fibrosis by cross-sectional histology of the hearts. Treatments were well tolerated throughout the in-life phase and no untoward effects on the physiology of the mice were noted. The following results demonstrate the activity of Compound 1 in heart failure models.

### Example 5

### Left Ventricle and Infarct Scar Remodeling in Mouse Model

The effect of Compound 1 on left ventricle and infarct scar remodeling was evaluated in a mouse model of permanent coronary artery occlusion. MI was created by permanently ligating the left anterior descending artery with a surgical suture. Treatments (QD, PO) were initiated 24 hours after MI and were continued for 28 days. Treatments consisted of suspension vehicle and Compound 1 at 0.3 mg/kg and 3 mg/kg. A cohort of mice that were subjected to the procedure but were not infarcted were included as a “sham operated” surgical control group. At the end of 28 days of treatment, the hearts were removed for histological evaluation of left ventricle structure and scar structure.

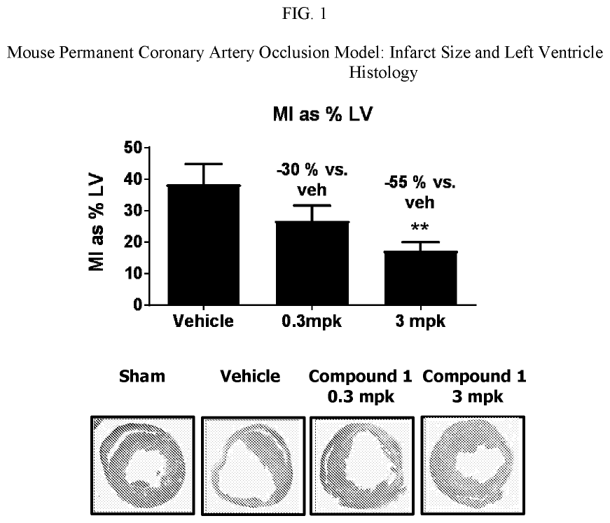

This is a model of ischemic heart disease. Infarct size was measured within the left ventricle (LV) and reported as a percentage of the total left ventricle cross sectional area. FIG. 1 shows a dose-dependent reduction in LV anterior wall infarct size with Compound 1 treatment where maximal reduction was obtained at the 3 mg/kg dose (55% vs. vehicle, P<0.01). Representative cross sections of trichrome-stained hearts for the various treatment groups and shams show that the extent of infarction and wall thinning is visually less in Compound 1 treatment groups than the vehicle group (FIG. 1 bottom panel).

### Example 6

### Effect of Compound 1 on Left Ventricle Wall Thickness

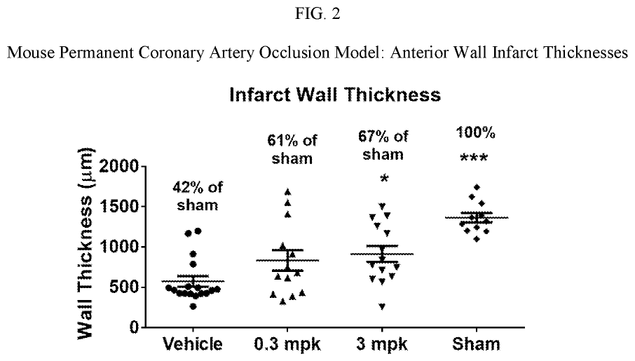

Treatment with Compound 1 preserved wall thickness relative to non-infarcted sham mice. Left ventricle wall thickness of sham mice represents the normal structure of the myocardial wall and thus serves as a reference for the post MI condition where scar expansion and thinning are expected. Wall thickness preservation was observed with both doses of drug: 61% of the mean thickness of sham at 0.3 mg/kg and 67% of sham at 3 mg/kg. Statistically significant differences vs. vehicle were achieved at the higher dose of Compound 1 (59%, P<0.05, FIG. 2). Left ventricle wall thickness of sham mice represents the normal structure of the myocardial wall and thus serves as a reference for the post MI condition where scar expansion and thinning are expected.

### Example 7

### Effect of Compound 1 on Left Ventricular Chamber Dilatation

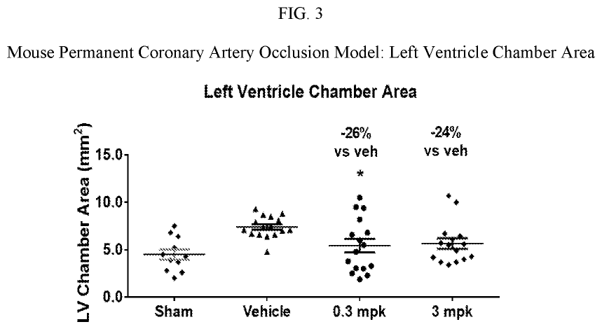

Left ventricular chamber dilatation was evaluated via measurements of cross-sectional area of the left ventricle cavity. Treatment with Compound 1 reduced LV chamber expansion at the 0.3 mg/kg dose (26% reduction vs. vehicle). A similar trend towards reduced chamber area was observed at the 3 mg/kg dose (24% reduction vs. vehicle, FIG. 3). Collectively, these data indicate less infarct expansion and left ventricle remodeling following MI.

### Example 8

### Changes in the Infarct Extracellular Matrix

In order to understand early changes in the infarct extracellular matrix, collagen remodeling enzymes and their endogenous inhibitors, the effects of 3 mg/kg Compound 1 treatment on the myocardial infarct composition in mice were evaluated 3 days post myocardial infarction. Myocardial infarction was created by permanently ligating the left anterior descending artery with a surgical suture. Treatment (QD, PO; suspension vehicle or Compound 1 at 3 mg/kg) was initiated 24 hours after MI and were continued to 3 days post MI. On day 3, mice received a final dose of treatment and the hearts were removed 2 hours post treatment (i.e., 74 hours after MI) for histological analysis.

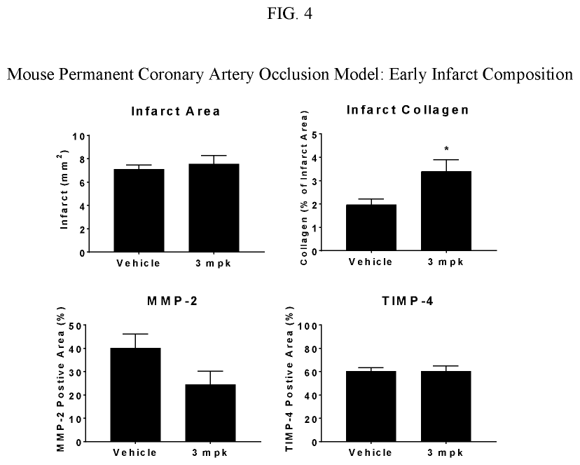

The top left graph of FIG. 4 shows infarct area measured in both treatment groups. As expected, average infarct areas between Compound 1 and vehicle treatments were similar and reflected an early stage of infarct prior to wall thinning and LV dilation. The top right graph shows the increased collagen content within the infarct of Compound 1-treated mice relative to vehicle (1.7-fold increased vs. vehicle, P<0.05), suggesting an early enhancement of collagen content. In addition, a trend towards less MMP-2 (matrix metalloproteinase-2) was detected in the infarcted myocardium vs. vehicle (bottom panel). The decrease in matrix remodeling enzyme levels are consistent with more infarct collagen in the Compound 1-treated group. No differences in tissue inhibitor of metalloproteinases-4 (TIMP-4), an endogenous inhibitor of MMPs, were detected in the infarcted tissue vs. vehicle (bottom panel). Taken together, these data suggest that early treatment with Compound 1 can help stabilize the infarcted myocardium via increased collagen content so as to lessen development of infarct wall thinning observed 28 days post MI.

In summary, Compound 1 was shown to favorably alter the progression of adverse remodeling of the left ventricle and myocardial scar that leads to heart failure following MI. The improvements in left ventricle and scar structure with Compound 1 treatment support the concept that FPR2 agonism can safely enhance wound healing following injury.

### Example 9

### Rat Model of Permanent Coronary Artery Occlusion

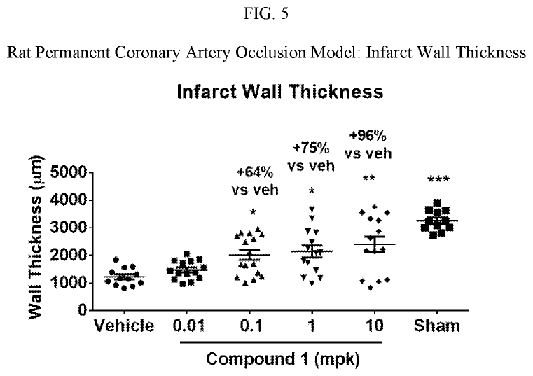

The effects of Compound 1 on left ventricle and infarct scar remodeling were evaluated in a rat model of permanent coronary artery occlusion. Treatments were initiated 48 hr after MI and were continued for 6 weeks. MI was created by permanently ligating the left anterior descending artery with a surgical suture. Treatments consisted of a suspension vehicle, Compound 1 at 0.01, 0.1, 1 and 10 mg/kg. A cohort of rats that were subjected to the procedure but were not infarcted were included as “sham operated” surgical control group. At the end of 6 weeks of treatment, hearts were removed for histological evaluation of left ventricle structure and scar structure. This is a model of ischemic heart disease. In addition, the effect of Compound 1 on cardiac function was measured by echocardiography to assess structure/function relationships. The structure of the myocardial wall was measured histologically. As shown in FIG. 5, treatment with Compound 1 resulted in a dose-dependent preservation of transmural infarct wall thickness, where maximal thickness was obtained at the 10 mg/kg dose (96% increase relative to vehicle, P<0.01). No changes in contralateral wall thicknesses were observed between treatment groups or non-infarcted surgical sham control rats.

### Example 10

### Measurement of Cardiac Function

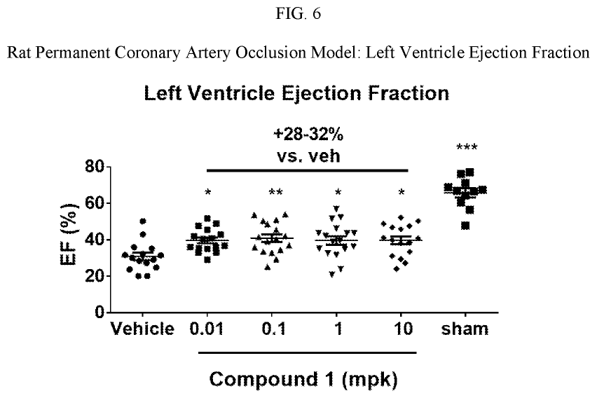

Echocardiography was used to measure cardiac function at the end of the treatment phase. The fraction of blood pumped out of the left ventricle per contraction cycle (i.e., ejection fraction) was determined using M-mode echocardiography. Improvements in left ventricular ejection fraction were obtained with Compound 1 treatments (28 to 32% vs vehicle, P<0.05, FIG. 6). All doses of Compound 1 yielded improvements in ejection fraction of a similar magnitude.

### Example 11

### Rat Model of Ischemia-Reperfusion Myocardial Infarction

The effects of Compound 1 on left ventricle and infarct scar remodeling were evaluated in a rat model of ischemia-reperfusion myocardial infarction. Treatments were initiated 48 hr after MI and were continued for 6 weeks. MI was created by transient ligation the left anterior descending artery with a surgical suture for 1 hour followed by reperfusion. Treatments consisted of a suspension vehicle, Compound 1 at 0.01, 0.1, 1 and 10 mg/kg. At the end of 6 weeks of treatment, hearts were removed for histological evaluation of left ventricle structure and scar structure. This is a model of ischemic heart disease.

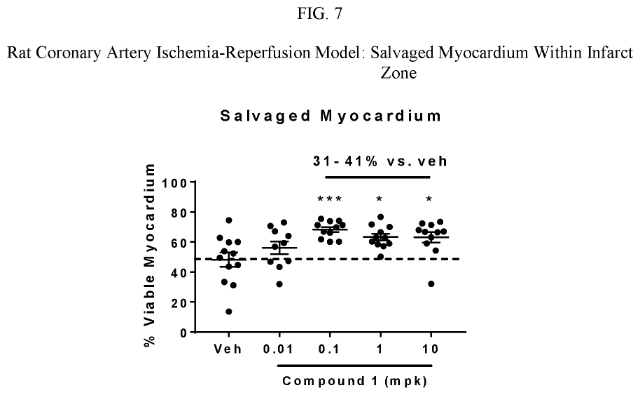

In addition, the effect of Compound 1 on cardiac function was measured by the pressure-volume conductance catheter technique to assess structure/function relationships. The structure of the infarct wall was measured histologically to determine the extent of myocardial infarction and myocardial tissue salvage. In this model, blood reperfusion to the site of ischemia is designed to salvage myocardium within the area at risk of myocardial infarction. However, in this model, there is potential for additional insult to tissue caused by reperfusion that can stimulate added myocardial necrosis. Therefore, measurement of myocardial salvage within the myocardial infarct zone is an important endpoint in this model. As shown in FIG. 7, treatment with Compound 1 resulted in an increase in myocardial salvage across the infarct wall, as measured by histology. Preservation of viable myocardium was obtained at the 0.1, 1 and 10 mg/kg doses (31-41% preservation relative to vehicle, *P<0.05, **P<0.01, ***P<0.001).

### Example 12

### Improvements in Left Ventricular Ejection Fraction

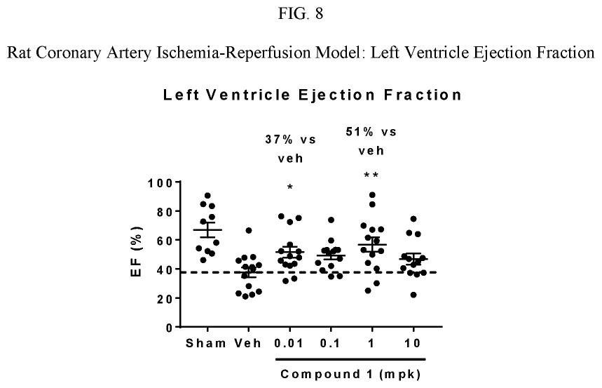

A pressure-volume conductance catheter was used to measure left ventricular ejection fraction at the end of the treatment phase. The fraction of blood pumped out of the left ventricle was measured per contraction cycle (i.e., ejection fraction) and was used to assess overall cardiac function following 6 weeks of treatment. Improvements in left ventricular ejection fraction were obtained with Compound 1 treatments (37% at 0.01 mg/kg vs. vehicle P<0.05 and 51% at 1 mg/kg vs vehicle, P<0.01, FIG. 8). It is worth noting a trend towards improved ejection fraction at the other doses vs. vehicle. These data are consistent with results obtained in the permanent coronary artery occlusion model.

In summary, Compound 1 was shown to favorably alter the remodeling of anterior wall MI via preservation of transmural infarct wall thickness and viable myocardium. These changes occurred parallel with improvements in LV ejection fraction measured by echocardiography and the pressure-volume conductance catheter technique. The improvements in scar structure and LV function with Compound 1 treatment support the concept that FPR2 agonism can safely enhance wound healing following injury.

### Example 13

### Angiotensin II-Induced Cardiac Fibrosis Mouse Model

Myocardial fibrosis was evaluated in the mouse with continuous angiotensin II challenge administered by subcutaneous osmotic mini-pump. This is a model of non-ischemic heart disease.

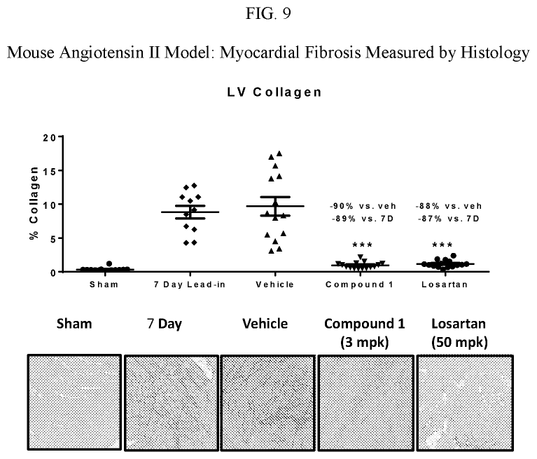

The effect of Compound 1 on regression of established cardiac fibrosis was evaluated in the angiotensin II-induced cardiac fibrosis mouse model. In this study, mice were challenged with angiotensin II for 7 days prior to starting treatment with Compound 1 in order to establish left ventricular fibrosis and evaluate regression of the condition. The angiotensin II receptor blocker, losartan, was used as a positive control for inhibition of sustained cardiac fibrosis. Since this animal model is driven by constant angiotensin II exposure, direct blockade of cognate receptor activation inhibits sustained fibrosis in the heart. As shown in FIG. 9, Treatment with 3 mg/kg of Compound 1 reduced left ventricular fibrosis by 90% when compared with vehicle treatment group (P<0.001). Moreover, when Compound 1 treatment was compared to the 7-day baseline group (a group of mice that was euthanized at 7 days to verify establishment of fibrosis), fibrosis was reduced by 89% relative to the pre-established fibrotic disease state (P<0.001). These data indicate that fibrosis can be regressed with Compound 1 treatment. Representative sections of left ventricular tissues depict the levels of interstitial fibrosis in the various group, as highlighted by the red stain.

### Example 14

### DOCA Challenged Cardiac Fibrosis Model

Myocardial fibrosis was evaluated in the mouse with continuous DOCA challenge administered by subcutaneous release pellet. The model increases systemic blood pressure and tissue inflammation in target tissues such as the myocardium via agonism of the mineralocorticoid receptor by DOCA. It also involves surgical uni-nephrectomy and supplementation with saline in the drinking water to further drive disease pathology. This is a model of non-ischemic heart disease.

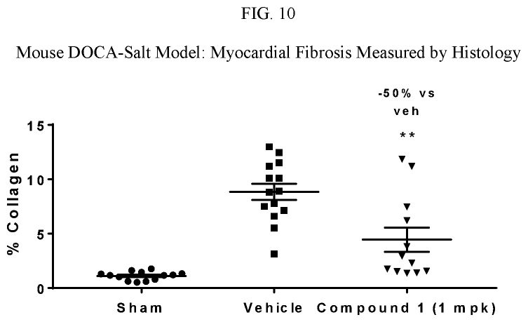

The effect of Compound 1 on cardiac fibrosis development in the DOCA-salt mouse model was evaluated histologically. In this study, mice were treated with Compound 1 for three weeks. Treatment started 24 hours after uni-nephrectomy and DOCA pellet implantation. As shown in FIG. 10, after 3 weeks of treatment with 1 mg/kg of Compound 1, left ventricular fibrosis was reduced by 50% when compared with vehicle treatment group (P<0.01). These data indicate that fibrosis development can be reduced with Compound 1 treatment.

It will be evident to one skilled in the art that the present disclosure is not limited to the foregoing illustrative examples, and that it can be embodied in other specific forms without departing form the essential attributes thereof. Reference being made to the appended claims, rather than to the foregoing examples, and all changes which come within the meaning and range of equivalency of the claims are therefore intended to be embraced therein.

We claim:

1. A method for the treatment of heart failure comprising administering to a patient in need thereof a therapeutically effective amount of Compound 1.

2. The method of claim 1, wherein the heart failure results from hypertension, an ischemic heart disease, a non-ischemic heart disease, exposure to a cardiotoxic compound, myocarditis, Kawasaki's disease, Type I and Type II diabetes, thyroid disease, viral infection, gingivitis, drug abuse, alcohol abuse, pericarditis, atherosclerosis, vascular disease, hypertrophic cardiomyopathy, dilated cardiomyopathy, myocardial infarction, atrial fibrosis, left ventricular systolic dysfunction, left ventricular diastolic dysfunction, coronary bypass surgery, pacemaker implantation surgery, starvation, an eating disorder, muscular dystrophies, and a genetic defect.

3. The method of claim 2 wherein the heart failure is selected from the group consisting of congestive heart failure, systolic heart failure, diastolic heart failure, heart failure with reduced ejection fraction (HF_{R}EF), heart failure with preserved ejection fraction (HF_{P}EF), acute heart failure, chronic heart failure of ischemic and non-ischemic origin.

4. The method of claim 3, wherein the heart failure is heart failure with reduced ejection fraction (HFᵣEF).

5. The method of claim 4, wherein the HF_{P}EF results from myocardial infarction.

6. The method of claim 5, wherein the compound is administrated after the diagnosis of myocardial infarction in the patient.

7. The method of claim 6, wherein the compound is administrated about 24 to 48 hours after the diagnosis of myocardial infarction in the patient.

8. The method of claim 6, wherein the compound is administered to the patient between about 1 and 7 days after the diagnosis of myocardial infarction in the patient.

9. The method of claim 3, wherein the compound is administrated orally.

10. The method of claim 3, wherein administration of the compound improves left ventricle function.

11. The method of claim 3, wherein administration of the compound prevents progression of myocardial wall thinning.

12. The method of claim 3, wherein administration of the compound inhibits cardiomyocyte cell death.

13. The method of claim 3, wherein administration of the compound reduces infarct size.

14. The method of claim 3, wherein administration of the compound reduces hypertrophy in the heart tissue.

15. The method of claim 1, wherein the compound is administrated daily.

16. The method of claim 1, wherein the compound is administrated for at least one, two, three, four, five, or six weeks.

17. The method of claim 3, wherein the heart failure is HF_{P}EF.

18. The method of claim 17, wherein the compound is administrated for at least one, two, three, four, five, or six weeks.

19. The method of claim 17 wherein administration of the compound improves myocardial wound healing.

20. The method of claim 17 wherein administration of the compound reduces ventricular fibrosis.

21. The method of claim 1, wherein the status of the condition of the patient's heart is assessed between each dose and the dose and/or timing of administration is adjusted in accord with the condition of the heart.

22. The method of claim 3, wherein the method comprises treatment of heart failure with a second agent.

23. The method of claim 22, wherein the second agent is selected from a diuretic, loop diuretic, a potassium sparing agent, a vasodilator, an ACE inhibitor, an angiotensin receptor blocker, an angiotensin II inhibitor, an aldosterone inhibitor, a positive inotropic agent, a phosphodiesterase inhibitor, a beta-adrenergic receptor inhibitor, a calcium channel blocker, an alpha blocker, a central alpha blocker, a sodium-glucose co-transporter 2 inhibitor, a nitrate, a statin, a cardiac glycoside, digoxin, nitrates, chlorthalidone, amlodipine, lisinopril, and doxazosin.

24. Compound 1 for use in the treatment and/or prophylaxis of heart failure.

25. The compound for use of claim 24, wherein said heart failure is HFᵣEF or HFₚEF.
